(19) United States
(12) Patent Application Publication
(10) Pub. No.: US 2014/0338668 A1
(43) Pub. Date: Nov. 20, 2014

(54) Respiratory Mask for Controlling Body Temperature and Method of Controlling Body Temperature by Using Respiratory Gas

(71) Applicant: U-BioMed Inc., Daegu (KR)

(72) Inventor: Nyeon Sik Eum, Daegu (KR)

(73) Assignee: U-BioMed Inc., Daegu (KR)

(21) Appl. No.: 13/894,517

(22) Filed: May 15, 2013

(51) Int. Cl. A61M 16/10; A61M 16/00; A61B 5/00; A61M 16/04; A61B 5/01; A61M 16/06; A61M 16/08

(52) U.S. Cl. CPC A61M 16/1095; A61M 16/0666; A61M 16/0057; A61M 16/0875; A61M 16/0051; A61M 16/0003; A61M 16/04; A61B 5/01; A61B 5/4836
USPC 128/204.15; 128/204.17

(57) ABSTRACT

A respiratory mask for controlling a body temperature of a patient by using a respiratory gas includes a controller for generating the respiratory gas, a gas transfer tube for receiving the respiratory gas and transferring the respiratory gas to a respiratory unit, a heat transfer metallic body configured to connect to the gas transfer tube and for cooling or heating the respiratory gas passing through the gas transfer tube, a thermoelectric element for performing an exothermic reaction or an endothermic reaction to heat or cool the heat transfer metallic body, a power supply unit configured to be located to be parallel to a bottom end of the gas transfer tube and for supplying power to the thermoelectric element via the controller, and a respiratory unit configured to connect to the gas transfer tube and for supplying the respiratory gas passing through the gas transfer tube to a patient.

### BACKGROUND

The present disclosure relates to a respiratory mask for controlling a body temperature, and more particularly, to a respiratory mask of cooling down or heating the air to be supplied to a patient and a temperature control device.

In general respiratory masks, a mask capable of covering the mouth or a respiratory organ such as the nasal cavity of a patient is put on and a respiratory gas generated by a controller is supplied through a gas transfer tube to the patient via the mask.

Such inhale way is incapable of supplying the air at a temperature appropriate for a body temperature of a patient.

Accordingly, there are used a method of using ultrasonic vibrations to control a temperature of a respiratory gas and a method of supplying steam by heating distilled water.

However, according to typical technology, a configuration for cooling down and heating a respiratory gas is complicated and the respiratory gas may be ineffectively transferred because a shape of a respiratory unit is not variable.

### BRIEF SUMMARY

Embodiments of the subject disclosure provide respiratory masks, as well as manufacturing and driving methods thereof, for controlling a body temperature by using a respiratory gas, the respiratory mask heating or cooling down the respiratory gas by using a thermoelectric element and cooling water and transferring the respiratory gas to lungs of a patient necessary to stable a body temperature thereof to easily control the body temperature.

Embodiments also provide respiratory masks for controlling a body temperature, which may efficiently supply a respiratory gas according to a condition of a patient by using various types of respiratory units.

In one embodiment, a respiratory mask controlling a body temperature by using a respiratory gas includes a controller generating the respiratory gas, a gas transfer tube receiving the respiratory gas from the controller and transferring the respiratory gas to a respiratory unit, a heat transfer metallic body connected to the gas transfer tube and cooling down or heating the respiratory gas passing through the gas transfer tube, a thermoelectric element performing an exothermic reaction or an endothermic reaction to heat or cool down the heat transfer metallic body, a power supply unit located to be parallel to a bottom end of the gas transfer tube and supplying power to the thermoelectric element via the controller, and a respiratory unit connected to the gas transfer tube and supplying the respiratory gas passing through the gas transfer tube to a patient.

In another embodiment, a method of controlling a body temperature of a patient by using a respiratory gas includes a controller, generating the respiratory gas, the controller, supplying the respiratory gas to a gas transfer tube via a gas insertion tube, a thermoelectric element, receiving power from a power supply unit and radiating heat or absorbing heat to control a temperature of the respiratory gas, heating or cooling down a heat transfer metallic body by providing heat or absorbing heat by the thermoelectric element, heating or cooling down the respiratory gas passing through the heat transfer metallic body connected to the gas transfer tube, transferring the heated or cooled down respiratory gas to a respiratory unit via the gas transfer tube, and supplying the respiratory gas discharged from the respiratory unit to the patient.

The details of one or more embodiments are set forth in the accompanying drawings and the description below. Other features will be apparent from the description and drawings, and from the claims.

### BRIEF DESCRIPTION OF THE DRAWINGS

FIG. 1 illustrates an oral and nasal type respiratory mask that is a respiratory mask for controlling a body temperature by using a respiratory gas according to an embodiment of the present disclosure;

FIG. 2 illustrates a nasal type respiratory mask that is a respiratory mask for controlling a body temperature by using a respiratory gas according to another embodiment of the present disclosure;

FIG. 3 is an oral-insertion type respiratory mask that is a respiratory mask for controlling a body temperature by using a respiratory gas according to still another embodiment of the present disclosure; and

FIG. 4 is a nasal-insertion type respiratory mask that is a respiratory mask for controlling a body temperature by using a respiratory gas according to yet another embodiment of the present disclosure.

### DETAILED DESCRIPTION OF THE EMBODIMENTS

As required, detailed embodiments of the present invention are disclosed herein. However, it is to be understood that the disclosed embodiments are merely exemplary and that various and alternative forms may be employed. The figures are not necessarily to scale. Some features may be exaggerated or minimized to show details of particular components. Therefore, specific structural and functional details disclosed herein are not to be interpreted as limiting, but merely as a representative basis for teaching one skilled in the art. The terminology used herein is for the purpose of describing particular embodiments only, and is not intended to limit the disclosure.

Reference will now be made in detail to the embodiments of the present disclosure, examples of which are illustrated in the accompanying drawings.

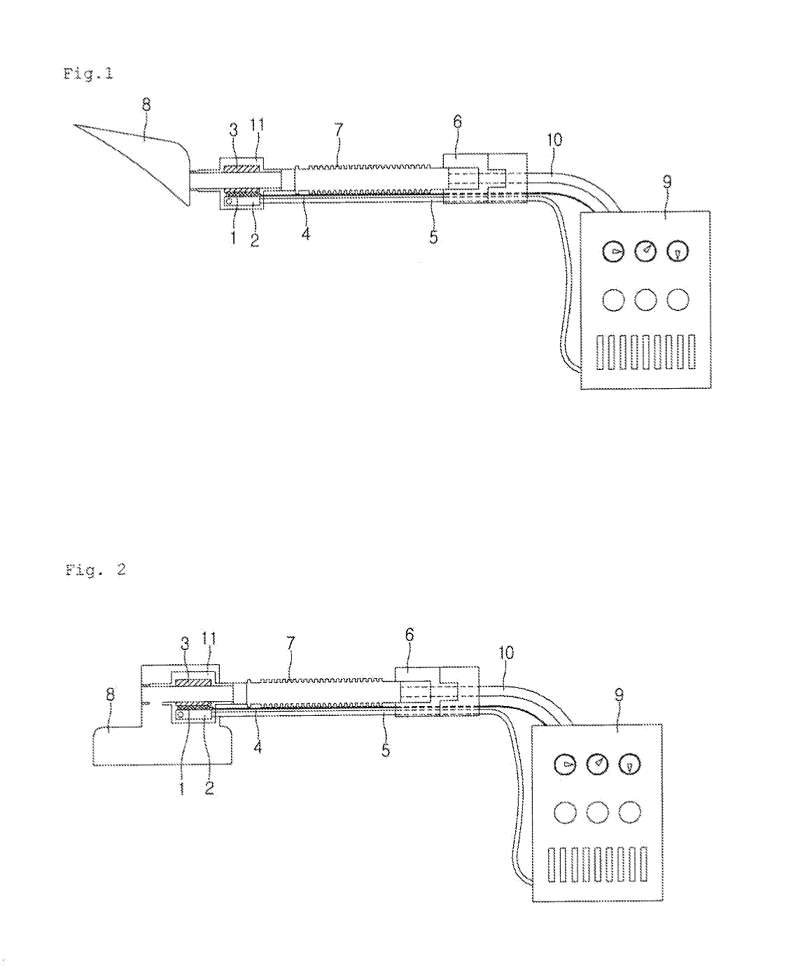

FIG. 1 is a mimetic view illustrating an oral and nasal type respiratory mask that is a respiratory mask for controlling a body temperature by using a respiratory gas according to an embodiment of the present disclosure.

Referring to FIG. 1, the respiratory mask according to the present embodiment includes a controller 9 generating a respiratory gas to be supplied to a patient, a gas insertion tube 10 connecting the controller 9 to a gas transfer tube 7, the gas transfer tube 7 transferring the respiratory gas to a respiratory unit 8, a thermoelectric element 1 providing heat or absorbing heat to control a temperature of the respiratory gas, a heat transfer metallic body 3 transferring the heat of the thermoelectric element 1 to the respiratory gas, a power supply unit 4 supplying power to the thermoelectric element 1, a thermoelectric element cooler 2 capable of cooling down the thermoelectric element 1, a cooling water tube 5 supplying cooling water to the thermoelectric element cooler 2, an insulating cover 11 for insulating the thermoelectric element 1 and the heat transfer metallic body 3 from the outside, and the respiratory unit 8 enfolding the mouth and the nose of the patient to supply the respiratory gas to the patient.

A configuration thereof will be described in detail as follows.

The controller 9 controls the entire configuration of the respiratory mask. In detail, the controller 9 directly generates or receives the respiratory gas from the outside and supplies the respiratory gas to the gas transfer tube 7 via the gas insertion tube 10. Also, the power supply unit 4 may supply power and the cooling water tube 5 may supply the cooling water. In this case, supply amounts of the respiratory gas and the cooling water may be controlled by the controller 9.

Also, the controller 9 may include a body temperature measuring unit and a temperature control unit. Accordingly, the controller 9 may monitor a real body temperature of the patient and a preset temperature by using the body temperature measuring unit and may control the temperature of the respiratory gas according to a change in the monitored body temperature by using the temperature control unit.

Also, one side of the gas transfer tube 7 is connected to the controller 9 via the gas insertion tube 10 and another side thereof is connected to the respiratory unit 8 to be a path of the respiratory gas. The gas transfer tube 7 may be formed of a wrinkled tube to allow the respiratory unit 8 to freely move.

Also, the thermoelectric element 1 is formed of a two kinds of metals, is located on one side of the heat transfer metallic body 3, and is connected to the power supply unit 4 to receive the power. The thermoelectric element 1 may provide heat or absorb heat to control the temperature of the respiratory gas. An endothermic reaction or an exothermic reaction of the thermoelectric element 1 is performed by using Peltier effect.

The Peltier effect denotes that, when two different metals are connected and a current is flowing therethrough, heat is generated or absorbed at a connection part of the two metals depending on a direction of the current. That is, according to the direction of the current, one of the two metals absorbs the heat and the other provides heat.

When receiving power from the power supply unit 4 in such a way that a current flow, the thermoelectric element 1 absorbs heat or provides heat according to the Peltier effect. The thermoelectric element 1 absorbing heat or providing heat heats or cools down the heat transfer metallic body 3, and the heat transfer metallic body 3 transfers heat to the respiratory gas to control a temperature of the respiratory gas.

Also, the heat transfer metallic body 3 is connected to the gas transfer tube 7 and may be formed of a metal having excellent heat conductivity such as aluminum and copper. The heat transfer metallic body 3 is cooled down or heated by the thermoelectric element 1, and the respiratory gas is heated or cooled down according to a temperature of the heat transfer metallic body 3 while passing through the heat transfer metallic body 3. That is, the heat transfer metallic body 3 transfers the heat of the thermoelectric element 1 to the respiratory gas.

Also, the thermoelectric element cooler 2 is connected to the thermoelectric element 1, the heat transfer metallic body 3, and the cooling water tube 5. The thermoelectric element cooler 2 may be formed of plastic that is resistant to corrosion caused by cooling water and is light in weight. The thermoelectric element cooler 2 receives cooling water from the cooling water tube 5 and lowers temperatures of the thermoelectric element 1 and the heat transfer metallic body 3.

On the other hand, the heat transfer metallic body 3, the thermoelectric element 1, and the thermoelectric element cooler 2 may be located to gas transfer tube 7 closed to the respiratory unit 8. A reason thereof is to quickly supply the respiratory gas at an appropriate temperature to the patient with no loss in temperature. However, it is not limited thereto.

Also, one side of the power supply unit 4 is connected to the controller 9 and another side thereof is located to be parallel to a bottom end of the gas transfer tube 7 and connected to the thermoelectric element 1.

The power supply unit 4 supplies power controlled by the controller 9 to the thermoelectric element 1.

Also, one side of the cooling water tube 5 is connected to the controller 9 and another side thereof is located to be parallel to a bottom of the gas transfer tube 7 and connected to the thermoelectric element cooler 2. The cooling water tube 5 supplies cooling water whose amount and temperature are controlled by the controller 9 to the thermoelectric element cooler 2.

On the other hand, the gas transfer tube 7, the power supply unit 4, and the cooling water tube 5 may be connected to the controller 9 through a connector 6 at a time.

Also, the insulating cover 11 may selectively enfold the thermoelectric element 1, the heat transfer metallic body 3, and the thermoelectric element cooler 2. The insulating cover 11 may be formed of a material capable of reducing thermal losses or a thermal inflow.

Also, one side of the respiratory 8 is connected to the gas transfer tube 7 and another side thereof is formed as a respiratory mask covering the mouth and the nose of the patient.

That is, the respiratory unit 8 receives the respiratory gas via the gas transfer tube 7 and supplies the respiratory gas to the patient via the respiratory mask.

A method of operating the elements described above will not be described as follows.

For example, a case in which the respiratory mask for controlling the body temperature operates when a warm respiratory gas is necessary for the patient will be described.

The controller 9 supplies power to the thermoelectric element 1 by using the power supply unit 4. In this case, the controller 9 may control a power supply amount and a power supply direction, that is, a direction of a current while considering the body temperature of the patient, a change in the body temperature, and a preset target value of the body temperature. The thermoelectric element 1 receiving the power provides heat, and the provided heat is provided to the heat transfer metallic body 3 connected to the gas transfer tube 7.

Also, the respiratory gas generated by the controller and passing through the gas insertion tube 10 and the gas transfer tube 7 passes through a heated heat transfer metallic body 3. In this case, the respiratory gas receives heat from the heat transfer metallic body 3 and a temperature thereof gets increased.

After that, the respiratory gas that gets warm is supplied to the respiratory unit 8 via the gas transfer tube 7 in such a way that the patient inhales the respiratory gas via a mask of the respiratory unit 8.

Also, a case in which a cool respiratory gas is needed will be described as an example.

The controller 9 supplies power to the thermoelectric element 1 by using the power supply unit 4. In this case, the controller 9 supplies a current in a way contrary to the method of supplying the warm respiratory gas. The thermoelectric element 1 receiving the power absorbs heat at the side connected to the heat transfer metallic body 3 to cool down the heat transfer metallic body 3. In this case, an opposite side to the side of the thermoelectric element 1, where an endothermic reaction occurs, is heated due to an exothermic reaction. Accordingly, cooling water supplied via the cooling water tube 5 may be allowed to pass through the thermoelectric element cooler 2 to lower temperatures of the heat transfer metallic body 3 and the thermoelectric element 1.

After that, the respiratory gas generated by the controller 9 and passing through the gas insertion tube 10 and the gas transfer tube 7 passes through a cooled heat transfer metallic body 3. In this case, since the heat transfer metallic body 3 absorbs heat of the respiratory gas, a temperature of the respiratory gas gets decreased.

After that, the respiratory gas that gets cool is supplied to the respiratory unit 8 via the gas transfer tube 7 in such a way that the patient inhales the respiratory gas via a mask of the respiratory unit 8.

Using the method described above, the respiratory mask for controlling the body temperature supplies the respiratory gas whose temperature is controlled according to the body temperature of the patient to be appropriate to the patient. In this case, the controller 9 may monitor a real body temperature of the patient and a preset temperature by using the body temperature measuring unit and may control the temperature of the respiratory gas according to a change in the monitored body temperature by using the temperature control unit.

FIG. 2 is a mimetic view illustrating a nasal type respiratory mask that is a respiratory mask for controlling a body temperature by using a respiratory gas according to another embodiment of the present disclosure.

Referring to FIG. 2, the respiratory mask for controlling the body temperature by using the respiratory gas is configured to form the shape of the respiratory unit 8 as the nasal type mask capable of covering only the nose. In the present embodiment, since the respiratory mask has the same configuration except the shape of the respiratory unit 8, the same elements are designated as the same reference numerals throughout.

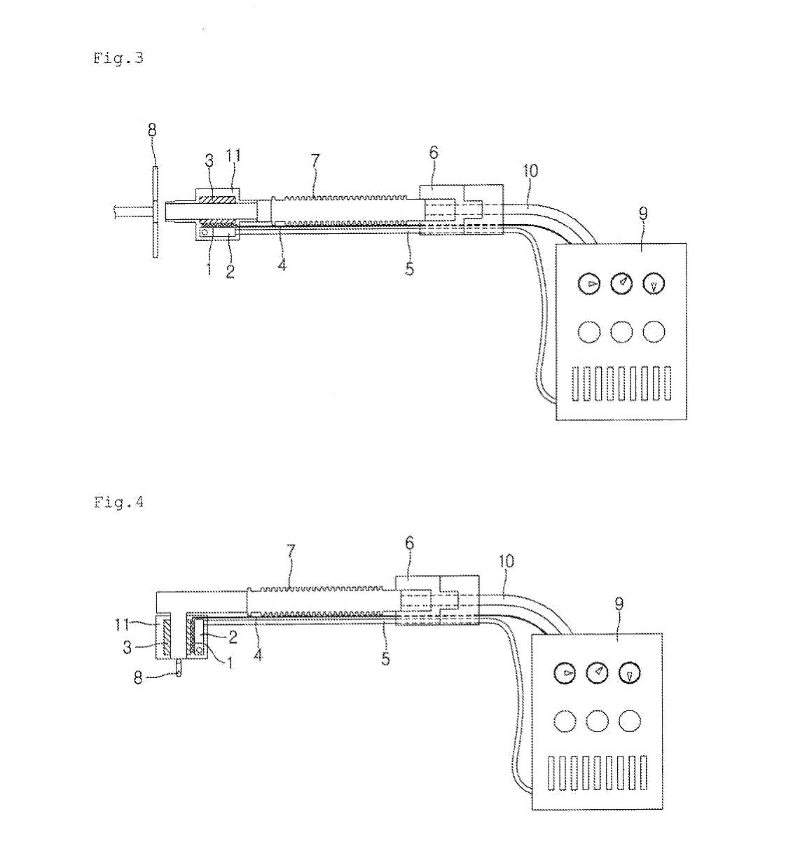

FIG. 3 is a mimetic view illustrating an oral-insertion type respiratory mask that is a respiratory mask for controlling a body temperature by using a respiratory gas according to still another embodiment of the present disclosure.

Referring to FIG. 3, the respiratory mask for controlling the body temperature by using the respiratory gas is configured to form a respiratory unit 8 as an oral-insertion unit capable of being inserted into the mouth. The shape of the respiratory unit 8 is a tube connected to the gas transfer tube 7 via a supporting plate to be inserted into the mouth of the patient. In the present embodiment, since the respiratory mask has the same configuration except the shape of the respiratory unit 8, the same elements are designated as the same reference numerals throughout.

FIG. 4 is a nasal-insertion type respiratory mask that is a respiratory mask for controlling a temperature by using a respiratory gas according to yet another embodiment of the present disclosure.

Referring to FIG. 4, the respiratory mask for controlling the body temperature by using the respiratory gas is configured to form the shape of the respiratory unit 8 as the nasal-insertion type mask capable of being inserted into the nose.

In the present embodiment, an end of the gas transfer tube 7 is bent downwards, the thermoelectric element 1 and the heat transfer metallic body 3 are installed on a bent part, and a tube is connected to the bent end of the gas transfer tube 7 to be inserted into nasal holes of the patient to be used as the respiratory unit 8 of the nasal-insertion type respiratory mask. In the present embodiment, since the respiratory mask has the same configuration except the shape of the respiratory unit 8, the same elements are designated as the same reference numerals throughout.

As described above, the respiratory unit 8 may include the oral and nasal type mask, the nasal type mask, the nasal-insertion type mask, and the oral-insertion type mask. According to a condition of the patient, one of the several examples of the respiratory unit 8 may be selected as the respiratory unit 8. Also, more variable shapes of the respiratory unit 8 may be formed.

According to the present embodiment, a temperature of a respiratory gas supplied to a patient may be controlled to control a body temperature of the patient.

Also, according to the present embodiment, since a respiratory mask may cool down or heat the respiratory gas by using a simple configuration, it is possible to supply the respiratory gas to the patient with no time delay.

Also, according to the present embodiment, various shapes of a respiratory unit may be provided according to a condition of the patient, thereby efficiently supplying the respiratory gas to the patient.

Any reference in this specification to “one embodiment,” “an embodiment,” “example embodiment,” etc., means that a particular feature, structure, or characteristic described in connection with the embodiment is included in at least one embodiment of the disclosure. The appearances of such phrases in various places in the specification are not necessarily all referring to the same embodiment. Further, when a particular feature, structure, or characteristic is described in connection with any embodiment, it is submitted that it is within the purview of one skilled in the art to effect such feature, structure, or characteristic in connection with other ones of the embodiments.

Although features have been described with reference to a number of illustrative embodiments, it should be understood that numerous other modifications and embodiments can be devised by those skilled in the art that will fall within the spirit and scope of the principles of this disclosure. More particularly, various variations and modifications are possible in the component parts and/or arrangements of the subject combination arrangement within the scope of the disclosure, the drawings and the appended claims. In addition to variations and modifications in the component parts and/or arrangements, various alternatives and alternative uses will also be apparent to those skilled in the art.

What is claimed is:

1. A respiratory mask for controlling a body temperature by using a respiratory gas, the respiratory mask comprising:
a controller for generating the respiratory gas;
a gas transfer tube for receiving the respiratory gas from the controller and transferring the respiratory gas to a respiratory unit;
a heat transfer metallic body configured to connect to the gas transfer tube and for cooling or heating the respiratory gas passing through the gas transfer tube;
a thermoelectric element for performing an exothermic reaction or an endothermic reaction to heat or cool the heat transfer metallic body;
a power supply unit configured to be located parallel to a bottom end of the gas transfer tube and for supplying power to the thermoelectric element via the controller; and
a respiratory unit configured to connect to the gas transfer tube for supplying the respiratory gas from the gas transfer tube to a patient.

2. The respiratory mask of claim 1, further comprising:
a cooling water tube configured to be located to be parallel to a bottom of the power supply unit and to connect to the heat transfer metallic body via the controller; and
a thermoelectric element cooler for receiving cooling water from the cooling water tube and for cooling the thermoelectric element and the heat transfer metallic body.

3. The respiratory mask of claim 2, further comprising a connector for connecting the gas transfer tube and the cooling water tube to the controller.

4. The respiratory mask of claim 1, further comprising an insulating cover for enfolding the thermoelectric element and the heat transfer metallic body.

5. The respiratory mask of claim 1, wherein the respiratory unit comprises a mask for covering the mouth and the nose of the patient.

6. The respiratory mask of claim 1, wherein the respiratory unit comprises a mask for covering only the nose of the patient.

7. The respiratory mask of claim 1, wherein the respiratory unit is configured to connect to the gas transfer tube and to be inserted into the mouth of the patient, and
wherein the respiratory unit includes a supporting plate to be connected to the gas transfer tube.

8. The respiratory mask of claim 1, wherein the respiratory unit comprises a mask capable of being inserted into nasal holes of the patient.

9. The respiratory mask of claim 1, wherein a part of the gas transfer tube comprises a wrinkled tube.

10. The respiratory mask of claim 1, wherein the controller comprises a body temperature measuring unit capable of measuring a body temperature of the patient and for monitoring the body temperature of the patient in real time.

11. The respiratory mask of claim 10, wherein the controller further comprises a temperature control unit for controlling a temperature of the respiratory gas, and
wherein the temperature control unit is configured to control the temperature of the respiratory gas by considering the body temperature of the patient and a preset target value.

12. The respiratory mask of claim 11, wherein the controller is capable of controlling a direction of the power, an amount of the power, and an amount of the cooling water to control the temperature of the respiratory gas.

13. The respiratory mask of claim 1, wherein the heat transfer metallic body comprises a metal having high heat transfer efficiency such as aluminum and copper.

14. The respiratory mask of claim 3, wherein the cooling water tube comprises a plastic.

15. The respiratory mask of claim 1, wherein the controller and the gas transfer tube are configured to be connected by a gas insertion tube.

16. The respiratory mask of claim 3, wherein the thermoelectric element, the heat transfer metallic body, and the thermoelectric element cooler are configured to be connected to the gas transfer tube closed to the respiratory unit.

17. The respiratory mask of claim 1, wherein the thermoelectric element comprises a Peltier element that absorbs heat or provides heat depending on a direction of a current from the power supply unit.

18. A method of controlling a body temperature of a patient by using a respiratory gas, the method comprising:
generating the respiratory gas via a controller;
supplying the respiratory gas to a gas transfer tube via a gas insertion tube;
radiating heat or absorbing heat to control a temperature of the respiratory gas via a thermoelectric element receiving power from a power supply unit;
heating or cooling a heat transfer metallic body by providing heat or absorbing heat by the thermoelectric element;
heating or cooling the respiratory gas passing through the heat transfer metallic body connected to the gas transfer tube;
transferring the heated or cooled respiratory gas to a respiratory unit via the gas transfer tube; and
supplying the respiratory gas discharged from the respiratory unit to the patient.

19. The respiratory mask of claim 18, further comprising, in cooling the heat transfer metallic body by absorbing heat by the thermoelectric element,
supplying cooling water to a thermoelectric element cooler via a cooling water tube to allow the thermoelectric element cooler to cool the thermoelectric element and the heat transfer metallic body.
